(19) United States
(12) Patent Application Publication
(10) Pub. No.: US 2009/0118629 A1
(43) Pub. Date: May 7, 2009

(54) RAPID METHOD FOR ANALYZING BIO-SIGNAL INSTANTANEOUSLY BY PHASE SPACE DIFFERENCE AND ITS DEVICE

(75) Inventors: Chii-Wann Lin, Taipei (TW); Tzn-Chien Hsiao, Shrlin Chiu (TW); Chien-Sheng Liu, Taichung City (TW)

Correspondence Address: SCHMEISER, OLSEN & WATTS, 22 CENTURY HILL DRIVE, SUITE 302, LATHAM, NY, 12110, US

(73) Assignee: NATIONAL TAIWAN UNIVERSITY, Taipei (TW)

(21) Appl. No.: 11/967,745

(22) Filed: Dec. 31, 2007

(30) Foreign Application Priority Data
Nov. 2, 2007 (TW) 096141581

(51) Int. Cl. A61B 5/0402

(52) U.S. Cl. 600/509

(57) ABSTRACT

The present invention relates to a method for analyzing bio-signal instantaneously by Chaotic Phase Space Difference (CPSD) operation and its measure analyze device. This method includes time-delay procedure and rebuilt phase space matrix to calculate chaos of phase space matrix and diagnose the bio-signal. This method can also be used as an analyze method in portable device or 24 h ECG recorder which is a fast and convenient measure analyze device.

### TECHNICAL FIELD

The present invention relates to a method and device for measuring, recording and analyzing bio-current signal of the human body, and particularly to a method and device for measuring, recording and analyzing bio-signal using Chaotic Phase Space difference analysis method.

### BACKGROUND OF THE INVENTION

The bio-signal could be used to evaluate and diagnose the important parameters for biological status, which employs the analysis on the bio-signal to be provided as the reference of clinical diagnosis. The bio-signal is characterized in having a periodically changing signal. The commonly used bio-signal includes the electrocardiogram (ECG or EKG), Heart Sound or Respiration Signal, which could be used to evaluate the cardiovascular system and lung function respiration system. The basic principles are briefly described as follows.

As shown in FIG. 1, the heart structure could be divided as two portions, the atrium and the ventricle, in which the atrium portion is connected with the upper and lower chamber veins. When the right atrium is full of the blood returned by the veins, the sinoatrial node (101) on the right atrium will spontaneously generate the depolarized action potential. The current signal will be transmitted to the left atrium through the muscle cells of the atrium. Because the muscle cells of the heart is provided with the ion channels suitable for electrical connection, the signal transmission is very fast, so that the left and right atriums will almost simultaneously depolarized, and further generate the contraction of muscle fibers, and generate the mechanical energy power to extrude the blood into the ventricle. At this time, the depolarized current signal will be transmitted to the atrio-ventricular node (102) at the bottom of the right ventricle. Because the signal transmission speed of the atrio-ventricular node is slower, the ventricle will have enough time to complete the operation of depolarized contraction. Next, the atrio-ventricular node will transmit the depolarized current signal to the entire left and right atriums through the Purkinje fibers (106), so the left and right ventricles are depolarized contraction simultaneously, and extrude the blood to the upper and lower chamber arteries, and accomplish a complete heart beat cycle. It could be noted that the heart employs the weak nerve current signal transmission to achieve the contraction and diastole action. Because the human body is a conductor, the current will conduct and flow all over the body through the human tissues. At this time, if attaching the conductible electrode patch on the body surface, it could employ the signal abscontraction circuit to record the current signal, and this signal is referred as the electrocardiogram (ECG or EKG) signal.

Generally in the ECG of so-called second leads body surface electrode record, the main signal composition is shown in FIG. 2A, which includes a P-wave representing the waveform signal measured and recorded on the body surface when the atrium is depolarized contraction, in which the measured and recorded is a QRS composite wave after about 0.15 seconds representing the depolarized contraction of the ventricle. At the same time, the ventricle will have the repolarized diastolic effect. But the repolarized signal strength of the ventricle is smaller than the depolarized signal strength, it could not be observed in ECG. The final T-wave represents the signal measured and recorded during repolarized diastole of the atrium. It could be found in the associated research, in various clinic diagnosis of diseases, the ECG will be appeared to present abnormal waveform or abnormal variation, such as ventricular hypertrophy, arrhythmia, myocardial infarction, coronary artery incompetence, and the like.

The heart sound signal is recorded with the sound given when the heart valve is closed. The most easily observed is the first heart sound (S1) and the second heart sound (S2), as shown in FIG. 2B. In the clinic, if the heart has the abnormal condition in the biological structure, besides of S1 and S2, there will be other murmur occurring. As shown in FIG. 2C, the signal occurred between S1 and S2 is the murmur, which is an important basis for determining heart disease.

Biologically, the speed of heart beat is controlled by various mechanisms, in which one of the important mechanisms is the respiration, and the speed of respiration will cause the variation of blood oxygen density, which will indirectly affect the heart rate. FIG. 2D shows the measurement result of the respiration signal.

In the method for analyzing bio-signal, the major domains have two portions: one is the analysis of frequency domain, which employs the fast Fourier Transform (FFT) to calculate the frequency spectrum of the bio-signal and observe the variance. For example, in the analysis of heart rate variability (HRV) for calculating the ratio of band energy of LF and HF, it is to observe the effect of the sympathetic nerve and the parasympathetic nerve to the heart rate variation; another one is to observe the waveform variance of the bio-signal, which is based on the analysis of Chaos Theory to understand the waveform distortion effect on the bio-signal caused by the disease, in which the commonly used analysis is the phase space matrix reconstruction. In the CPSD (Chaotic Phase Space Difference) algorithm, it employs the calculation of CPSD to generate the reference data for determining the bio-signal. For the application of ECG, it first could be used to calculate the heart rate, which has replaced the conventional R-R interval calculation method, and effectively solved the problem of threshold value selection in R-R interval calculation, and it could further easily determine the normal and abnormal ECG signal. In the application of heart sound, it could employ the CPSD algorithm to distinguish S1 and S2 to differentiate the murmur, and calculate the heart rate instantaneously. In the application of respiration signal, the CPSD algorithm could be used to calculate the variance of respiration rate.

In the PCT Patent No. 2004023995 published on Mar. 25, 2004, it disclosed a device and method for measuring subcutaneous ECG waveform through the R-wave algorithm. The device is mainly used for implanted defibrillator or inserted loop recorder, and employs the interval difference between R-wave and R-wave to determine if arrhythmia has occurred and as the basis of recording and defibrillating. In the calculation of measurement method, employing the R-wave algorithm and the automatic threshold value regulation method to precisely abstract the R-wave message as the basis of calculation of interval difference between R-waves.

Although using the interval difference between R-waves as the measurement method for ECG has been disclosed in the content of the prior art, using the interval difference between R-waves as the ECG measurement method will be limited by the selection of the threshold, which could not easily and rapidly differentiate the difference between normal and abnormal ECG signals. In order to solve this problem, it is required an ECG analysis method for easily editing, fast processing speed, saving the storage space, and reducing consumed system resources.

### SUMMARY OF INVENTION

The present invention provides a rapid method for analyzing bio-signal by CPSD and the measurement and analysis device. The object is to overcome the defects of the bio-analysis method described in the prior art for consuming more system resources and wasting much time on determination for not achieving instantaneous analysis. The CPSD analysis method employed by the present invention is based on the following steps to proceed the bio-signal analysis:

1. With the Following Steps to Establish the Phase Space Matrix:

A. abstracting the bio-signal, and after filtering out the unnecessary noise by the filter, selecting the suitable maximum signal amplitude and applying the normalization on the amplitude; and, employing the maximum amplitude to define the size of the rebuilt phase space matrix, and initializing the phase space matrix to configure the initial value as zero;

B. on the signal time axis, selecting the original as the datum point, and selecting the suitable time interval from the datum point as the reference point;

C. employing the bio-signal strength at the datum point and reference point to demarcate the two coordinates of the phase space matrix, and accumulating the values at the location of the coordinates;

D. sequentially adding the datum point and the reference point; repeating Step C until all the bio signals are processed.

2. With the Following Steps to Rebuild the Phase Space Matrix to Obtain the Chaotic Phase Space Difference:

A. selecting suitable parameter configuration, including data length, time interval, sampling rate, size of phase space matrix, and delay time, and the like;

B. establishing the phase space matrix of reference bio-signal, which is referred as a reference matrix in brief;

C. establishing a phase space matrix for analyzing the bio-signal, which is referred as an analysis matrix in brief;

D. establishing a phase space matrix for storing the bio-signal, which is referred as a result matrix in brief;

E. calculating the variance of the label points of two space matrixes, and the calculation employs the analysis matrix shown in Step C and the reference matrix shown in Step B; because the sized of the two matrixes are the same, they could be directly conducted with subtraction operation; and, the operation will subtract the values of matrix elements in the analysis matrix from the values of matrix elements with the same coordinates in the reference matrix; and, the subtraction result is stored in the same coordinate positions in the result matrix; and, after completion of subtracting each coordinate element in the matrix, calculating the data with the positive value in the result matrix, and the calculate data is the chaotic phase space difference; and, employing the mean of CPSD value and the variance of standard deviation, which could automatically suitably adjust the threshold and the scope thereof, and the calculation is the mean plus/minus three times of standard deviation.

3. Differentiating Normal and Abnormal ECG Signal:

Based on the chaotic phase space difference, it will select the suitable threshold range as the basis of differentiation. When the CPSD value exceeds the scope, it will be determined as an abnormal ECG signal, as shown in FIG. 3A. The curve A (solid circle) indicates the variance curve for CPSD value obtained by the calculation with CPSD algorithm; the curve B (phantom circle) and the curve C (solid inversed triangle) indicate the upper limit and the lower limit of the threshold scope, respectively. When the CPSD value is within the threshold range, the ECG signal establishing the phase space matrix will be determined as normal. When the CPSD exceeds or is lower than the threshold range, the ECG signal establishing the phase space matrix will be determined as abnormal. As shown in FIG. 3B, the solid line indicates the ECG signal abstracted and loaded for analysis; the phantom line indicates the record result, and the zero value indicated the normal ECG signal. For example, the highest value 400 in FIG. 3B indicates the abnormal ECG signal. It could be found that the ECG signal could be differentiated with the abnormal portion of the premature ventricle contraction by successfully and completely labeled by the CPSD analysis method.

4. Determining if the Heart Sound Signal has Occurred Murmur:

Employing the CPSD value to calculate the mean and standard deviation (SD) of CPSD value in fixed length, as shown in FIG. 3C; the solid line indicates the CPSD value variance curve calculated with the CPSD algorithm, and the thin phantom line and the thick phantom line indicate the mean and the SD, respectively; when there is not murmur in the heart sound signal, the SD will be larger than the mean; when the murmur is appeared, as shown in FIG. 3D, the mean will be larger than the SD; and, it could be found that the central noise portion of the heart sound signal could be successfully and completely distinguished with the CPSD analysis method.

5. Determining the Speed Variation in Respiration Signal:

The CPSD value could be used to indicate the variance of the respiration speed. As shown in FIG. 3E, the phantom line indicates the CPSD value variance curve calculated with the CPSD algorithm, and the solid line indicates the respiration signal, which could be found that when the difference of respiration speed becomes larger, the relative CPSD value will also be increased.

### BRIEF DESCRIPTION OF DRAWINGS

FIG. 1 is a cross-sectional diagram of heart structure;

FIG. 2A is an ECG signal cycle recorded by the body surface electrode;

FIG. 2B is the normal heart sound signal;

FIG. 2C is the heart sound signal with murmur;

FIG. 2D is a respiration signal;

FIG. 3A is the determination of CPSD value signal between normal and abnormal ECG;

FIG. 3B is the normal and abnormal ECG signals;

FIG. 3C is the CPSD value, the mean and the SD for normal heart sound signal;

FIG. 3D is the CPSD value, the mean and the SD for murmur signal;

FIG. 3E is the respiration signal and the CPSD value curve;

FIG. 4 is the relationship between heart rhythm and CPSD;

FIG. 5A is a reference matrix established with the normal ECG signal;

FIG. 5B is an analysis matrix established with the normal ECG signal;

FIG. 5C is a result matrix established with the normal ECG signal;

FIG. 6A is a reference matrix established with the abnormal ECG signal;

FIG. 6B is an analysis matrix established with the abnormal ECG signal;

FIG. 6C is a result matrix established with the abnormal ECG signal; and,

FIG. 7 is a flow diagram for establishing the phase space matrix.

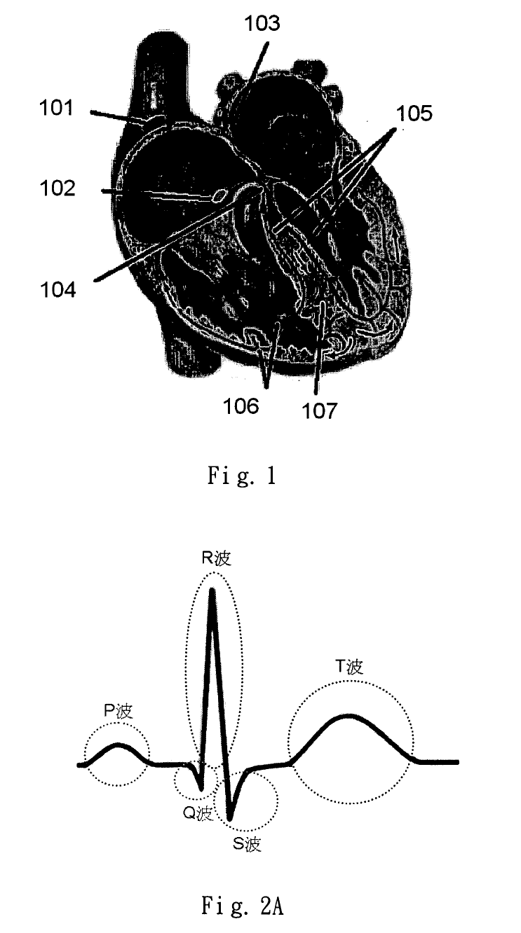

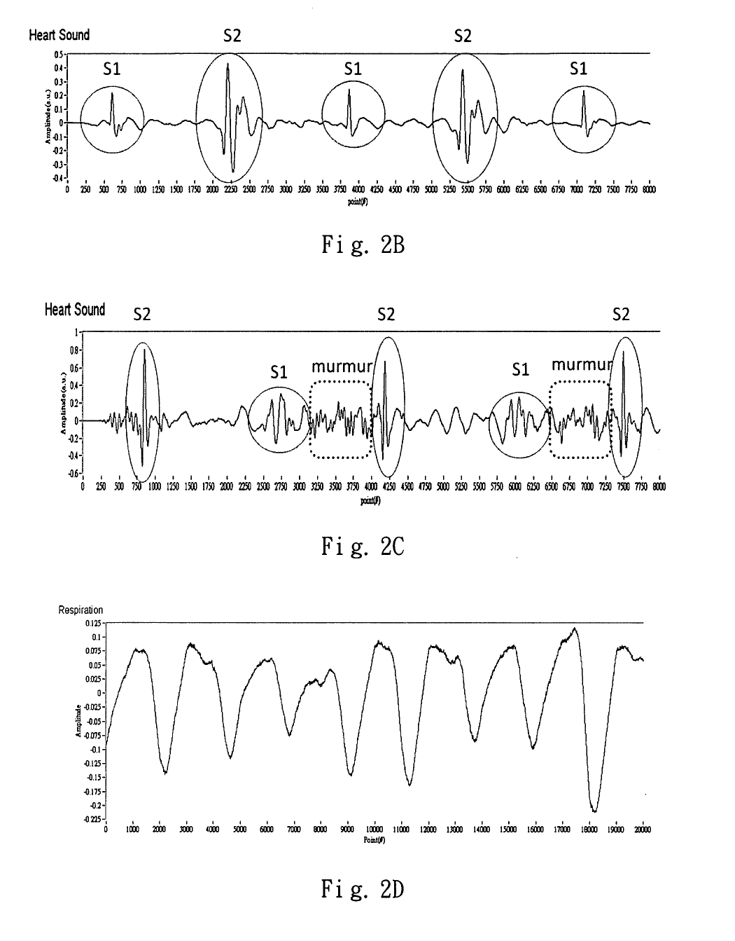

### DETAILED DESCRIPTION OF PREFERRED EMBODIMENTS

In order for the examiners to understand the objects, the features and the effects of the present invention, the following embodiments associated with the attached figures will be described in details for the present invention as follows. The present embodiment employs the ECG signal analysis as an example, but the same analysis model could be applied to the bio-signal with periodical variance.

1. Flow Chart of Embodiment for Calculating CPSD:

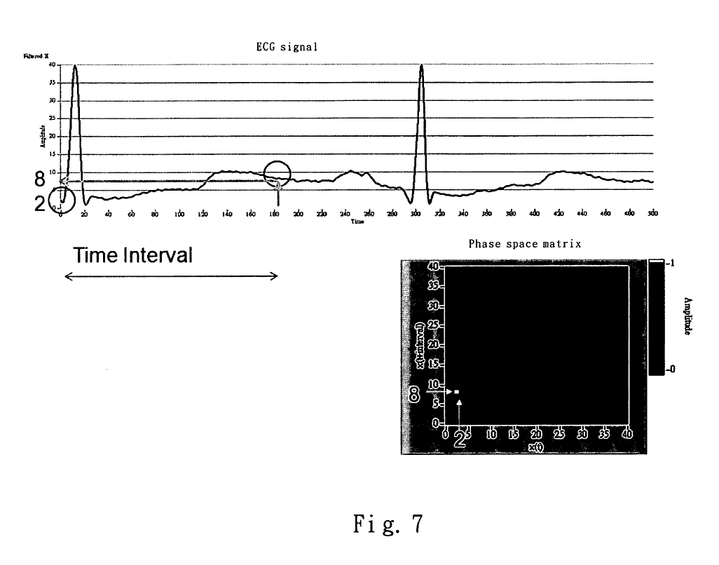

Employing the ECG signal abstraction device to abstract the ECG signal with the preferred sampling rate at 250˜500 Hz; the signal after abstraction will be with suitable data length to establish the phase space matrix with the preferred data length as 5˜10 seconds; this section of flow for establishing the phase space matrix using ECG signal is shown in FIG. 7; for the initialization portion of the phase space matrix, the size of the matrix is the same as the normalization value of the ECG. The size of phase matrix in FIG. 7 is 40×40, so the maximum value of ECG signal after amplitude normalization is 40 with the preferred normalization parameter is 20˜50, and the initial values for the elements in the phase space matrix after initialization is configured as zero; next, employing the ECG signal after normalization to establish the phase space matrix, which the datum point is started from the original of the time coordinate; then, obtaining the coordinates of reference points after selecting suitable time interval with the preferred time interval at 0.2˜1 seconds. The coordinate for reference point shown in FIG. 7 is 180. From the ECG signal after normalization, it could be known the signal strength at the coordinate of the datum point is 2, and the corresponding signal strength at the coordinate of the reference point is 8, so as to obtain a set of coordinates (2, 8); at this time, adding one to the contents of element labeled with coordinates (2, 8) in the phase space matrix; next, adding one to the coordinates of the datum point; and, the new coordinate of the datum point is the position of time axis 1; using the same time interval to obtain the new coordinates of the reference point, which is the position of the time axis 181; labeling a set of coordinates composed with ECG signal strength corresponding to the two coordinates in the phase space matrix and adding one to the content; and, repeatedly executing this step until this section of ECG signal is processed completely.

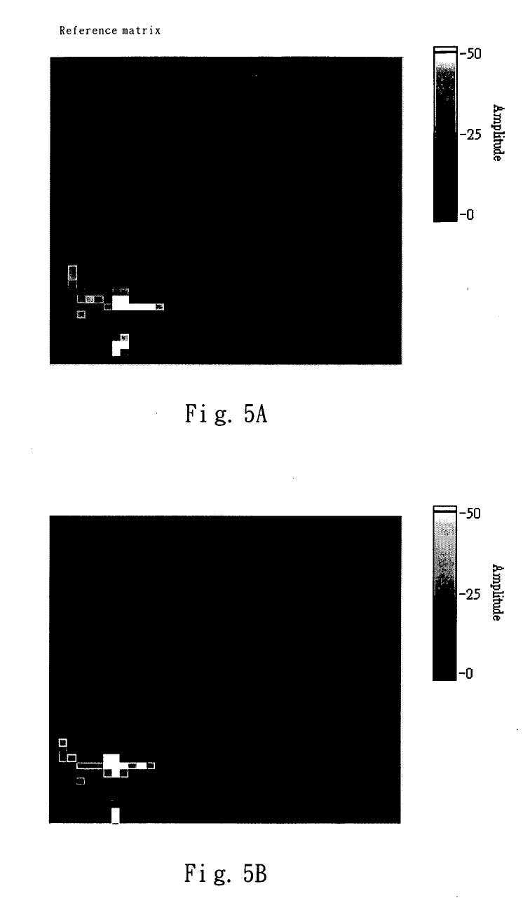

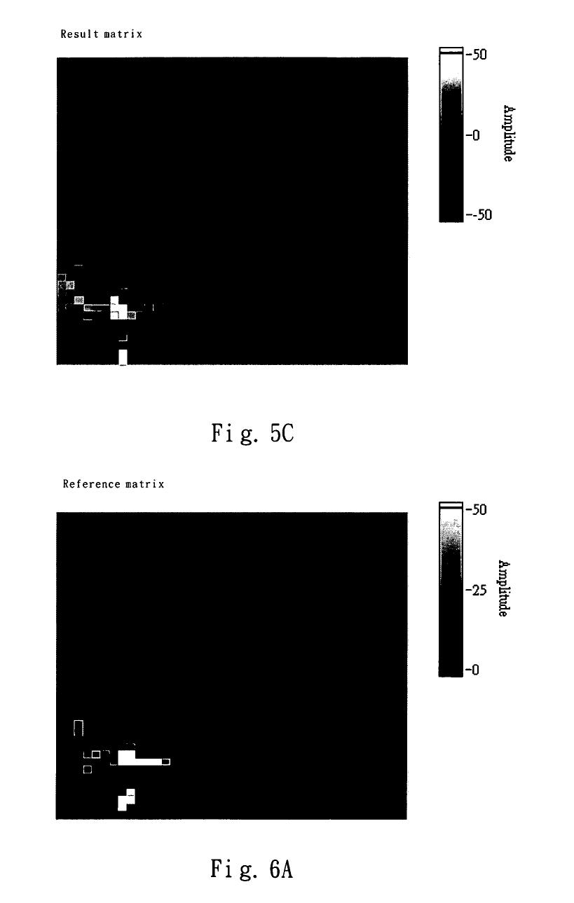

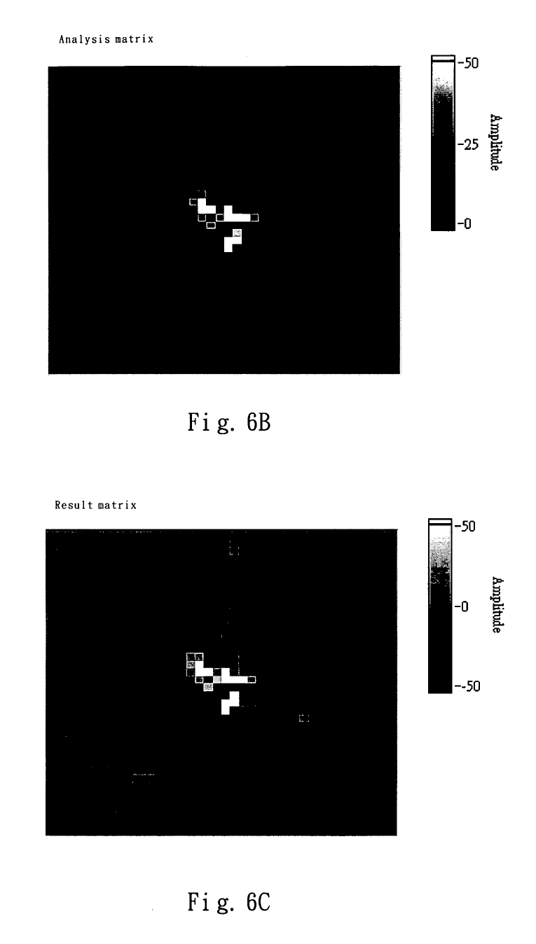

If the abstracted ECG signal is used as a reference, the generated phase space matrix is a reference matrix. FIGS. 5A and 6A are the reference matrixes representing normal and abnormal ECG signals, respectively; if the abstracted ECG signal is used for analysis, the generated phase space matrix is an analysis matrix. FIGS. 5B and 6B are the reference matrixes representing normal and abnormal ECG signals, respectively.

By subtracting the contents of the analysis matrix from the contents of the reference matrix, the difference between two matrixes could be obtained, and the subtraction result is stored in the result matrix. FIGS. 5C and 6C are the result matrixes representing normal and abnormal ECG signals, respectively. It could be found in FIGS. 5C and 6C that the result after subtraction includes a positive value and a negative value. In this algorithm, the calculation result by counting the number of data with positive value in the matrix is the CPSD value.

2. Differentiating the Normal and Abnormal ECG Signals:

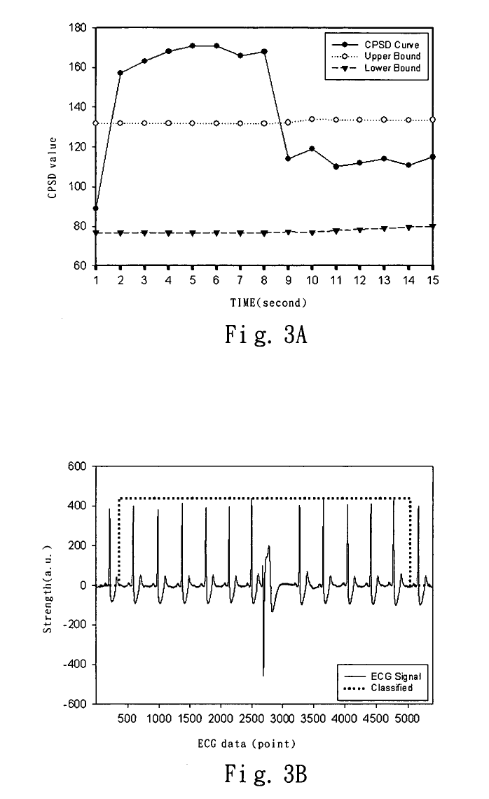

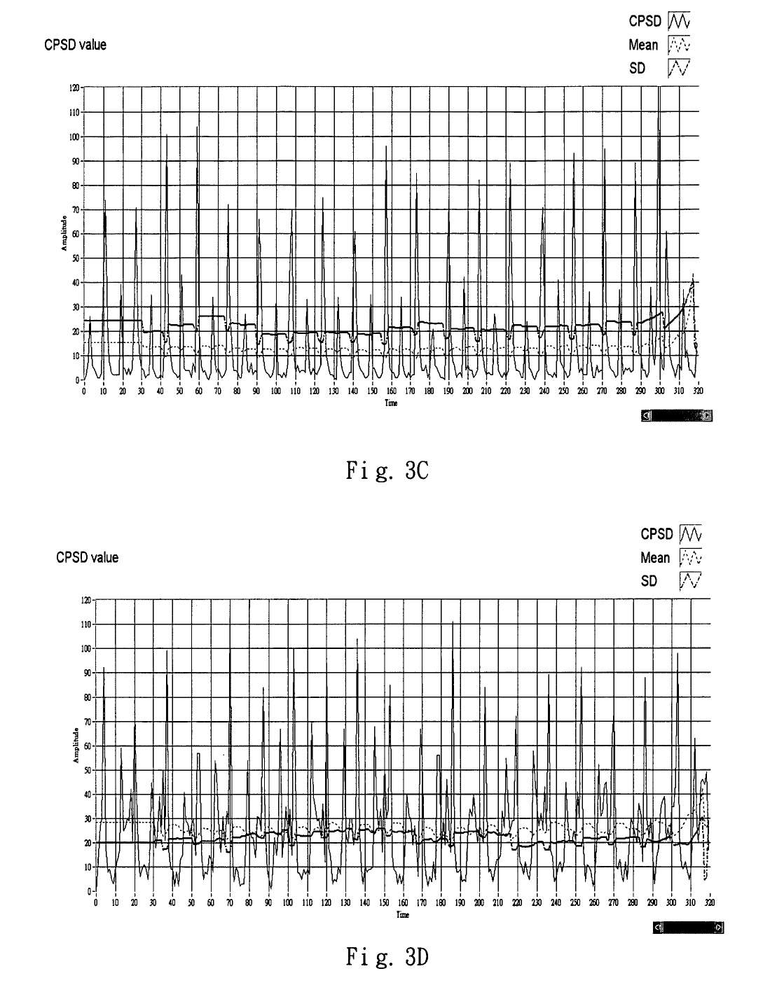

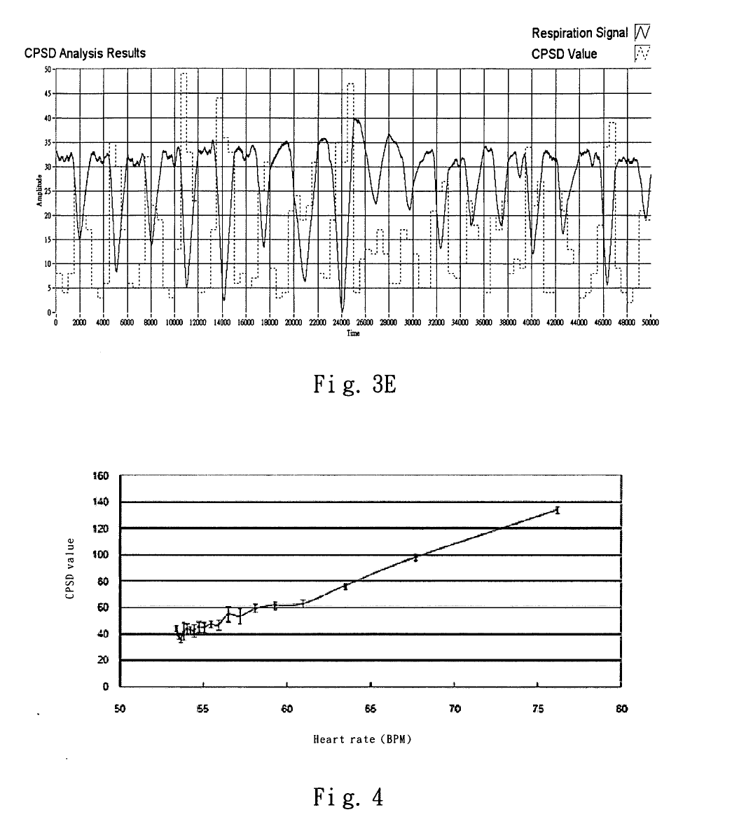

Employing the CPSD, it could select the suitable threshold range as the basis for differentiation. When the CPSD exceeds the range, it would be determined as an abnormal ECG signal. As shown in FIG. 3A, the curve A indicates the variance curve of CPSD obtained using CPSD algorithm, and the curve B and the curve C indicate the upper limit and the lower limit of the threshold range, respectively. When the CPSD is within the threshold range, the ECG signal for establishing the phase space matrix at this time will be determined as normal. When the CPSD exceeds or is lower than the threshold range, the ECG signal for establishing the phase space matrix at this time will be determined as abnormal. As shown in FIG. 3B, the solid line indicates the ECG signal abstracted and loaded for analysis, and the phantom line indicates the record result, and the zero indicates the normal ECG signal. For example, the highest value 400 in FIG. 3B indicates the abnormal ECG signal. It could be found that the ECG signal could be differentiated with the abnormal portion of the premature ventricle contraction by successfully and completely labeled by the CPSD analysis method.

3. Calculating the Heart Rate of Normal ECG Signal:

In the CPSD analysis method, when CPSD is located within the threshold range, CPSD could be used to calculate the corresponding heart rate. The relationship between heart rate and CPSD shown in FIG. 4 could be found that when the heart rate is larger than 62 (BPM), the heart rate and the CPSD will exhibit with a very good linear relationship. Moreover, the variation range (standard deviation) for CPSD will not have overlapped effect. Thus, the CPSD could be used to calculate the corresponding heart rate as the reference for other determination.

4. ECG Signal Analysis Result in Arrhythmia Database (BIH-MIT):

The table below represents the ECG signals for different diseases in BIH-MIT based on the determination result using PSD analysis method, and each data length is for 30 minutes, and the sampling rate is 360 Hz; wherein, V indicates the Premature Ventricular Contract, A indicates the Atrial premature contraction, a indicates the aberrated atrial premature, F indicates the Ventricular fusion beat, and VT indicates the Ventricular Tachycardia.

| | Used/Total | | | | |
|---|---|---|---|---|---|
| Database | Memory | | | | |
| Record | Space | catched | loosed | Sensitivity | Hint |
| 101 | 0.09 | 4 | 5 | 0.44 | A |
| 103 | 0.38 | 2 | 0 | 1.00 | A |
| 106 | 0.53 | 513 | 7 | 0.99 | V |
| 113 | 0.10 | 5 | 1 | 0.83 | a |
| 116 | 0.69 | 108 | 2 | 0.98 | V, A |
| 123 | 0.10 | 3 | 0 | 1.00 | V |
| 205 | 0.38 | 85 | 0 | 1.00 | VT, V, F, A |

By embedding the CPSD analysis method into the microprocessor, it could be used for ECG analysis in the following devices:

(1) Standalone 24-hour ECG recorder;

(2) Portable apparatus for instantaneously measuring, analyzing and recording ECG signal, such as PDA and cell phone;

(3) Improvement on the performance of the existed ECG measurement and analysis device; and,

(4) Integrated ECG measurement and analysis system composed by combining the transmission interface.

5. The Preferred Range and the Optimized Value for the Parameters Used in CPSD Algorithm:

Using CPSD algorithm to analyze the bio-signal must be configured with the value range for associated parameters according to different bio-signal characteristics. Based on the result of experimental analysis, the preferred range and the optimized value for the associated parameters used in the related bio-signal analysis is provided for the reference in implementation.

a. ECG Signal:
i. Sampling rate: the preferred range is 250˜500 Hz, and the optimized value is 360 Hz;
ii. Data length: the preferred range is 5˜10 seconds, and the optimized value is 7 seconds;
iii. Normalized parameter: the preferred range is 20˜50, and the optimized value is 40;
iv. Time interval: the preferred range is 0.2˜1 seconds, and the optimized value is 0.2 seconds; and,
v. Delay time: the preferred range is 5˜10 seconds, and the optimized value is 7 seconds.

b. Heart Sound Signal:
i. Sampling rate: the preferred range is 5 k˜10 kHz, and the optimized value is 8 kHz;
ii. Data length: the preferred range is 10˜50 ms, and the optimized value is 25 ms;
iii. Normalized parameter: the preferred range is 20˜50, and the optimized value is 40;
iv. Time interval: the preferred range is 1˜2 ms, and the optimized value is 1.25 ms; and,
v. Delay time: the preferred range is 10˜50 ms, and the optimized value is 25 ms.

c. Respiration Signal:
i. Sampling rate: the preferred range is 250˜500 Hz, and the optimized value is 500 Hz;
ii. Data length: the preferred range is 5˜10 seconds, and the optimized value is 7 seconds;
iii. Normalized parameter: the preferred range is 20˜50, and the optimized value is 40;
iv. Time interval: the preferred range is 0.2˜1 seconds, and the optimized value is 0.2 seconds; and,
v. Delay time: the preferred range is 5˜10 seconds, and the optimized value is 7 seconds.

What is claimed is:

1. A rapid method for analyzing bio-signal instantaneously by phase space difference, which is characterized in comprising the following steps:
(A) selecting suitable parameter configuration;
(B) establishing one phase space matrix for reference and another phase space matrix for analyzing bio-signal;
(C) calculating the variance condition for the label points between two space matrixes to obtain the variance of chaotic phase space difference; and,
(D) evaluating whether the chaotic phase space difference exceeds the threshold range or not for determining if the bio-signal is normal.

2. A method for analyzing bio-signal according to claim 1, wherein the parameters configured in Step (A) includes the data length, the time interval, the sampling rate, the normalized parameters, and the delay time.

3. A method for analyzing bio-signal according to claim 1, wherein the establishing method for reference and analyzing phase space matrixes in Step (B) includes the following steps:
(a) eliminating the noise interference in the bio-signal;
(b) employing the parameter of the size of the phase space as the basis of normalization parameters;
(c) conducting normalization process on the amplitude of the bio-signal;
(d) employing the normalization parameters to configure the size of the space matrix, and initializing the contents in the matrix as zero;
(e) configuring the origin coordinates as the coordinates of the datum point;
(f) employing the parameter of time interval and the datum point to configure the coordinates of the reference point;
(g) employing the strength of the bio-signal at the datum point and the reference point to label the two coordinates of the phase space matrix, and adding the values in the location of the coordinates; and,
(h) sequentially adding the datum point and the reference point to establish the phase space matrix.

4. A method for analyzing bio-signal according to claim 1, wherein the method for calculation of variance at labeled points between two space matrixes to obtain the chaotic phase space difference in Step (C) includes the following steps:
(a) employing the method for establishing the phase space matrix and the reference bio-signal to establish the reference matrix;
(b) employing the method for establishing the phase space matrix and the analysis bio-signal to establish the analysis matrix;
(c) employing the parameter of the size of the phase space to establish the result matrix;
(d) employing the subtraction operation to calculate the difference between each element value in the analysis matrix and the reference matrix, and store the operation result in the result matrix; and,
(e) counting the number of elements with positive value in the matrix, and the counting result is the chaotic phase space difference.

5. A method for analyzing bio-signal according to claim 1, wherein the method for employing whether the chaotic phase space difference exceeds the threshold range as the standard for determining if the bio-signal is normal in Step (D) includes the following steps:
(a) applying statistical analysis on suitable data number of the chaotic phase space difference, and calculating the mean and the standard deviation;
(b) employing the mean and the standard deviation to calculate the threshold range; and,
(c) configuring the threshold range as the means plus/minus three times of the standard deviation.

6. A method for analyzing bio-signal according to any one of claim 2 or 4, wherein the bio-signal is an ECG signal, a heart sound signal, a respiration signal, or other bio-signals with periodicity.

7. A method for analyzing bio-signal according to claim 6, wherein when the bio-signal is an ECG signal and a respiration signal, the optimized range for suitable parameter configuration in Step (A) includes the followings:
(a) Data length: 5˜10 seconds;
(b) Time interval: 0.2˜1 seconds;
(c) Sampling rate: 250˜500 Hz;
(d) Normalization parameter: 20˜50; and,
(e) Delay time: 5˜10 seconds.

8. A method for analyzing bio-signal according to claim 6, wherein when the bio-signal is a heart sound signal, the optimized range for suitable parameter configuration in Step (A) includes the followings:
(a) Data length: 10˜50 ms;
(b) Time interval: 1˜2 ms;
(c) Sampling rate: 5 k˜10 kHz;
(d) Normalization parameter: 20˜50; and,
(e) Delay time: 10˜50 ms.

9. A bio-signal measurement and analysis instrument, which is characterized in that the device employs the method for calculating the chaotic phase space difference according to claim 4 as the analysis method for bio-signal.

10. A bio-signal measurement and analysis instrument according to claim 9, wherein the bio-signal is an ECG signal, a heart sound signal, a respiration signal, or other bio-signals with periodicity.

11. A bio-signal measurement and analysis instrument according to claim 10, wherein the device is provided with an embedded module with function for calculating the chaotic phase space difference.

12. A bio-signal measurement and analysis instrument according to claim 10, wherein the device could be used for 24 hours in a standalone manner.

13. A bio-signal measurement and analysis instrument according to claim 10, wherein the device may be a portable apparatus for instantaneously measuring, analyzing and recording the bio-signal.

14. A bio-signal measurement and analysis instrument according to claim 10, wherein the device is an integrated bio-signal measurement and analysis system composed by combining a transmission interface.
